(19) United States
(12) Patent Application Publication
(10) Pub. No.: US 2008/0234719 A1
(43) Pub. Date: Sep. 25, 2008

(54) Vaginal dilator for use in vaginal rehabilitation and methods therefor

(76) Inventor: Justin Bruce Adams, Louisville, KY (US)

Correspondence Address: JUSTIN BRUCE ADAMS, 913 Washburn Ave., Louisville, KY, 40222, US

(21) Appl. No.: 12/079,035

(22) Filed: Mar. 24, 2008

Related U.S. Application Data
(60) Provisional application No. 60/896,457, filed on Mar. 22, 2007.

(51) Int. Cl. A61M 29/00; A61F 2/04

(52) U.S. Cl. 606/193; 606/191; 623/23.7

(57) ABSTRACT

These stents aid in the rehabilitation of transgender patients, vaginismus patients and vaginal reconstruction patients and similar maladies. The glass stent is made from select medical grade borosilicate glass that is non-porous, hypoallergenic, incredibly durable, and incredibly smooth, can be easily sterilized, is lightweight, and greatly reduces friction during removal. By being hollow, the typical suction effect that is created during removal is eliminated as air can enter the vaginal cavity. There is a tapered hour glass area and an open and lipped end so insertion and removal is more easily facilitated. There are also three indentions in the glass as depth guides for the patient that can be both seen and felt but are very smooth. Finally, this stent can be tailored to individual patients if their bodies require different measurements.

### CROSS-REFERENCE TO RELATED APPLICATION

This application claim priority to U.S. provisional application 60896457, filed Mar. 22, 2007.

### BACKGROUND OF THE INVENTION

This invention pertains to the art of methods and apparatus used for stents, also commonly referred to as vaginal dilators, for use in vaginal rehabilitation patients. The stent maintains the shape of the vaginal wall for conditions such as transgender surgery, vaginismus, dyspareunia, radiotherapy, post childbirth, and for patients having psychological inhibitions to intercourse, but no limited to these afflictions.

The basic purpose of the stent is to maintain the shape of the vaginal cavity. For some, the stent prevents reduction in diameter of the vaginal cavity while others use the stent to increase the cavity diameter in cases where it is too narrow. Patients begin with a daily therapy regimen and eventually switch to weekly therapy which can last their entire lives. The stent is inserted for a few hours a day, removed and then sanitized. Patients require a stent that can be comfortably and securely inserted, does not interact or adhere with vaginal tissue, is lightweight, durable, easily sterilized, and can be removed without difficulty.

Vaginal stents in the prior art, typically, have a pointed tip giving it an overall bullet-like shape. Some have deep squared grooves cut perpendicularly into the stent and others simply use small notches that do not encircle the stent. The lengths and points at which these indentations occur vary. The deep squared grooves are easily visible and felt by the patient but the vaginal wall can constrict into these grooves. Upon removal, this creates a problem of additional friction and discomfort.

The prior art structural features used to aid in the removal of the stent have two basic designs. On the proximal end of a prior art stent, some designs use a wide flange base which acts both as a stop guide and as a handle. It is generally much wider than the stent and provides a good area for gripping. However, a known problem exists when the bases of the larger diameter stents prevents the patient from closing their legs completely. This restricts their overall movement and comfort. Other designs finish the proximal end with a perpendicular cut across the material leaving a shear face. In some designs, a separate grabbing tool is used and inserted into the proximal end for gripping purposes. The need for the small gripping tool creates known issues for patients with dexterity problems. This can lead to anxiety and stress with the patient. Deep grooved designs generally do not need the additional tool as the grooves provide some small level of grip.

Stents are used as a set with multiple sizes, and a complete set will usually include four or five stents ranging in size from 25 mm to 38 mm. Each individual stent is about 3 or 4 mm larger than the preceding one, although not limited to these dimensions. This may be undesirable to some patients who use their stents to expand the vaginal cavity. Increasing the diameter by 3 mm will increase the stent circumference by over 9 mm, resulting in additional discomfort to the patient. In cases where children are involved, smaller diameter stent sizes are not readily available.

Stents in the prior art are typically made from medical grade silicones and plastics, and mainly are deformable materials. Although smooth to the touch, on a microscopic level, these materials are very porous. During therapy, the vaginal wall will naturally constrict around the stent, forming a tight seal. The microscopic pores of these materials allow the vaginal wall to grip the stent. The resulting friction prevents easy removal of the stent and adds potential discomfort and stress. These porous materials can also harbor bacteria and microorganisms. The patient has to take special care to properly sterilize the stent before each use.

In the area of sterilization, the porous materials require thorough cleaning and care to prevent contamination. Although proper cleaning methods are given with each stent, some patients still feel anxiety on whether the stent is sterilized or not, due to the porous nature of these materials. A non-porous material is optimal.

Stents in the prior art usually are solid in design, but this creates internal pressure problems. During insertion, air in the vaginal cavity needs to escape as the volume of the stent replaces it. Although use of lubrication aids the release of the air, there is still some internal pressure that pushes against the stent as it is inserted. Then during removal, after the vaginal muscles have constricted around the stent and a tight seal has formed between the stent and vaginal wall, air needs to enter the cavity to replace the volume previously occupied by the stent. The less air that enters the cavity the more internal backpressure exists. This backpressure restricts the stent from being easily removed. A design where the air can easily flow is preferred.

### SUMMARY OF THE INVENTION

A borosilicate glass vaginal dilator that is hollow to allow air to move freely, that facilitates secure gripping and handling for insertion and removal, that provides both tactile and visual depth guides, is durable, lightweight and easily sterilized, is one preferred embodiment.

An advantage of the present invention is the hollow, tubular design which alleviates internal air pressures that occur during insertion and removal by allowing air to flow without restriction into and out of the vaginal cavity.

Another advantage of the present invention is the slightly indented depth markers that are both visually and tactilely noticeable around the entire circumference of the stent, while being completely smooth without sharp edges.

Another advantage of the present invention is the hourglass neck formed between the body and the retractor that provides an area for secure gripping between the fingers by reducing the diameter of the dilator allowing two fingers of the patient to surround and squeeze this reduced portion of the invention thus providing enough friction to easily control and guide the dilator.

Another advantage of the invention is the open caudal end which allows a patient to insert a fingertip into this portion of the dilator to enable more secure handling. The overall structure of the caudal end allows the patient to pinch the open end between their fingers.

Yet another advantage of one preferred embodiment of the invention is the thick inner lip on the open caudal end which further enhances the gripping ability of the inserted fingertip by creating a soft edge that the tip of the finger can grab onto and hold.

Still another advantage of one preferred embodiment of the present invention is it being integrally formed of medical grade borosilicate glass material. The material, when formed, is completely non-porous which reduces friction, reduces the amount of lubricant needed, and is easily cleaned and sterilized. Borosilicate is chemically inert so patients will not have any interactions with the material. It is incredibly strong and durable while being lightweight. It holds its temperature very well which allows it to be pre-warmed before use reducing the temperature gradient between patient and dilator.

Another advantage of the present invention is that the dilator, or a set, is individually made and can therefore be quickly customized to a patient's requests such as a change in overall length, specific diameter requests, or where the depth markers will be placed, but not limited to these changes.

Another advantage of the invention is the manufacturing equipment and tool setup does not change for each different diameter or length of dilator regardless of what size was made previously.

Another advantage of the invention is the availability of many different diameters with increments of 1 mm, starting at 14 mm all the way to 40 mm, but not limited to these sizes. This wide variety of available sizes give the patient more control over the size and will reduce the stress on the patient as the switch to larger diameter dilators and allow them better control of the size when they are preventing further reduction of the vaginal cavity.

Another benefit derived from the structural features of the invention is the hollow design which reduces the weight of the material used and thus reduces the internal discomfort a patient feels due to the weight of the stent.

### BRIEF DESCRIPTION OF THE DRAWINGS

FIG. 1 is a distal end view of a borosilicate glass vaginal stent or dilator of the invention;

FIG. 2 is a caudal end view of the article;

FIG. 3 is a side elevation cross sectional view of the device taken generally along lines 3-3 of FIG. 1; and

FIG. 4 is a side perspective view of the article.

### DETAILED DESCRIPTION OF THE INVENTION

The present invention is a stent, or dilator, to aid in vaginal rehabilitation. The dilator helps to maintain vaginal cavity shape. The dilator is used both to prevent the further reduction of the vaginal cavity and to help expand the cavity in cases where it is too narrow.

The invention embodied in FIG. 3 is produced using a glass working torch and a lampworking lathe, but is not limited to this equipment, as these can be made by hand and using different types of gas torches. The preferred embodiment of the invention depicted in FIG. 3 is made entirely from borosilicate glass. Some steps used to make the article are known in the art, but the combination of steps described hereinafter are novel. The torch generally uses an oxygen-propane combination but it not limited to these components. The overall process remains the same even if the equipment changes. The lampworking lathe is a machine that has two chucks to hold the glass, instead of a person using their hands to hold and rotate the glass. Each is controlled by a motor that rotates the two chucks simultaneously. One chuck is remains stationary at all times while the second chuck can be repositioned along the axis of rotation of the lathe so the distance between the chucks can be shortened or lengthened. Moving the second chuck away from the first elongates the glass while moving them closer will compress the hot glass. When a lathe is not used, arms and hands will accomplish these tasks.

To begin forming the article, we start with select borosilicate glass tubing, referred to as a tube, and visually inspect it for areas where the manufacturing process has left scratches or nicks on the surface and subsequently reject any glass that is not visually ideal. The glass is held up to a light source which will visually show areas on the glass where it is scratched or nicked. This borosilicate glass material is preferred but structural equivalents of the invention could be formed from metals, wood, and some plastics. Once the tubing has passed visual inspection, it is mounted into the holding chucks of the lathe. With the lathe on and the glass tube rotating at a moderate speed, we begin by using a soft, low heat intensity flame directed perpendicular to the glass to prevent it from heating too quickly and collapsing into itself. A soft flame is one where more propane is used and there is no a strong hissing sound of oxygen coming from the torch. Generally, this is a low temperature flame.

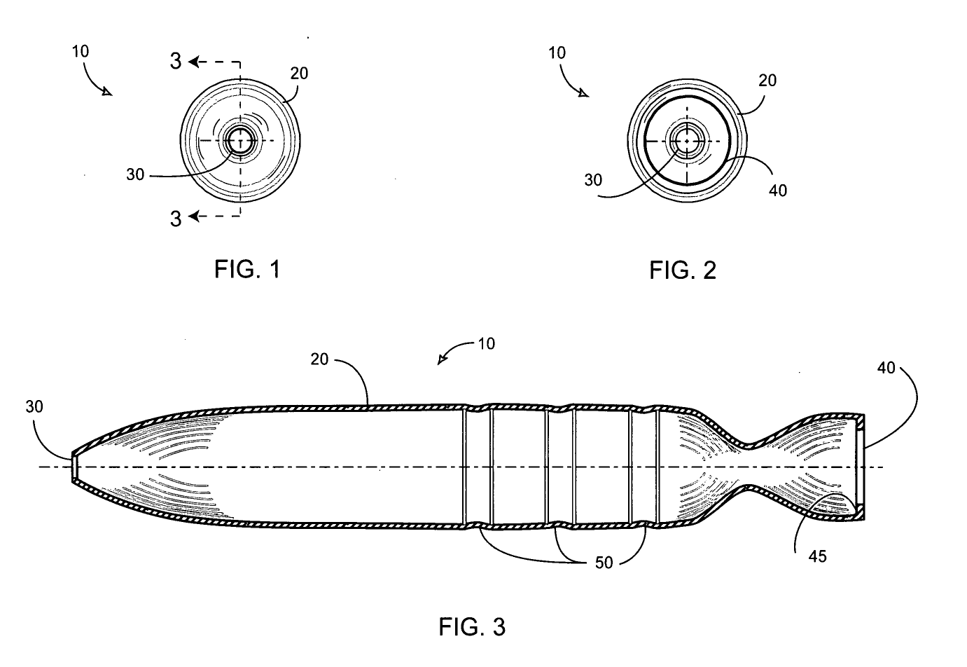

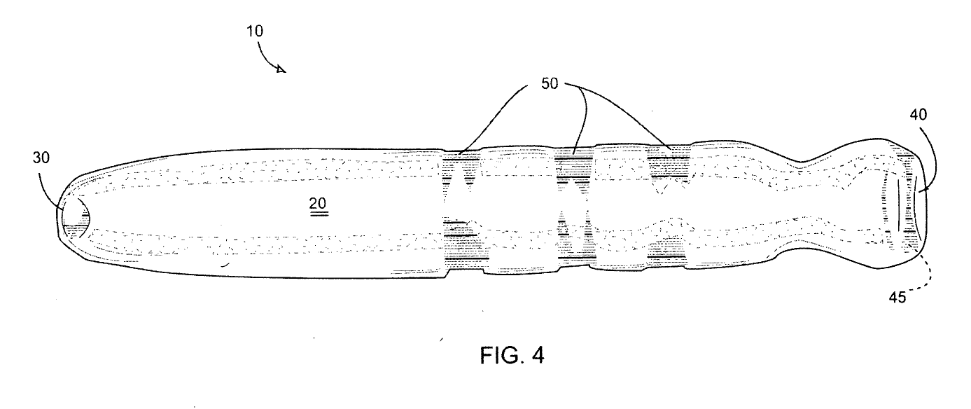

It does not matter if the process steps are started by working from the right side or from the left side of the material. The process starts at a point at least two inches in and away from the tip of the lathe chuck to keep the chuck from heating up too much. This can cause the glass to crack if the expansion rate of the glass is significantly different from the expansion rate of the metal of the chuck. The flame is slowly moved back and forth and is concentrated over an area about one inch wide. As the glass heats it starts to turn orange and become soft. When the glass is malleable but not molten the artisan slowly widens the adjustable chuck so the glass will stretch and lengthen, at the same time the diameter of the glass over this area reduces and starts to form the tapered distal end in FIG. 3. Adjust the location of the flame and the chuck to create a tapered slope in the glass that is slightly larger than the finished diameter. When the glass tube is stretched to create the taper the thickness of the tube wall is reduced. To restore the wall thickness to its original size, we use a soft low heat flame over the tapered portion until it begins to slowly condense upon itself. The diameter along the taper is minutely reduced as the thickness of the tube wall is increased. The narrowest point of the taper should be at least 10 mm to leave space for the hollow tip, or port, 30. Make sure that the diameter of the tube at the smallest point of the taper is at least half of the overall tube diameter, leaving space to form the hole or pressure port 30 in FIG. 1.

When the shape of tapered distal end is achieved, continue to use the soft low heat flame and gradually heat the glass and move slowly down its length to the distance where the depth guides begin. The flame must not be moved to quickly along the tube as not to stress and fracture it but it must be moved quick enough to prevent the tube from condensing upon itself.

To form the indented depth markers 50 in FIG. 3, measure from the smallest point of the tapered tip to a point approximately 3 mm past the point where the marker is to be centered and concentrate the heat on that point. Use a small needlelike flame no more than a few millimeters in diameter. As the glass becomes very soft it begins to droop or sink inward and condense upon itself forming an indentation, or very small hourglass shape. Adjust the moveable chuck closer to compress the glass which at this point increases the wall thickness and expands the indention outward and forms a hump making it wider than the tube.

Continue to use the needlelike flame and concentrate it on this hump. Heat this area with the small flame until the hump starts to condense inward and its diameter nears the starting diameter of the tube. Just before the hump diameter reduces to match the tube diameter remove the flame to slow the process with the goal of having the markers diameter 50 in FIG. 3 only be one millimeter smaller than the overall starting tube diameter. When finished the depth marker 50 will have a very slight indentation in the glass that is very smooth but is noticeable to the touch. In addition, the build up of the wall thickness of the glass at the marker results in a noticeable visual ring contained within the stent. To make additional markers 50 in FIG. 3, the process is simply repeated at the required placements. These markers can also be reduced or increased in numbers or even eliminated if preferred.

These markers may be created by wrapping a thin colored glass stringer around the clear tube and melting it into the clear tube. This method is not preferred as colored borosilicate has slightly different physical properties due to the minerals used to create each color and thus affects the beneficial properties of the clear glass. There is also the risk that a bubble may get included during the process which can lead to a weak point within the glass. To add the colored stringer, we would measure to the exact point from the distal end where the marker should be located. We adjust the flame of the torch so that only 10-15% of it is touching the invention. The majority of it is above the tube. With the flame in front of the art hold the tip of a colored glass rod and heat it in the flame until the first few millimeters are molten. With the invention rotating, touch the tip of the colored rod to the art at the point where the marker should be located. A stringer will pull from the rod and wrap around the glass, a thin and uniform stringer is preferred. The flame is used to cut the stringer from the artwork and the colored rod is removed from the flame and set aside. The entire flame is refocused on the area where the stringer has been laid and it is slowly melted to artwork to create a small indention. Heat is applied until the colored stringer has fused with the clear invention. A very needlelike flame is then used to allow the area of the colored stringer to reduce slightly inward to create the tactile reference guide. An overall reduction in diameter of one millimeter at this marker is preferred. In cases where other materials are used, these depth markers can be displayed using inks to print the marker on the outer surface, although this will leave no tactile reference for the patient.

Next, to form the hourglass neck, we adjust the flame back to the softer less intense flame used previously. We measure four millimeters past the center point of the neck and concentrate this softer flame on this point until the glass begins to condense inward. When the diameter is reduced to three-fourths of the original, we adjust the moveable chuck very slightly, only one or two millimeters, to help stretch and condense the hourglass shape of the neck. We remove the flame and let the glass continue to condense until the diameter of the hourglass neck is around half of the original tube diameter completing the neck.

To form the open caudal end 40 and inner lip 45, as depicted in the FIG. 3 embodiment, measure one inch past the hourglass neck and center the flame. Adjust the flame back to a needlelike flame used previously. Focus the flame on this point until the glass starts to condense inward at which time we slowly widen the moveable chuck as we did for the tapered distal end. In this case we keep widening the chuck until the glass completely condenses inward and closes the tube on the caudal end. With the caudal end sealed and the flame still focused on the point of closure, we continue to widen the chucks until the glass separates into two pieces, the partially formed dilator with a closed caudal end and the remaining piece of raw material.

To make the open end 40 and the inner lip 45, we use a needlelike flame again focused directly into the center of the sealed caudal end, not on the edges. The flame is oriented almost be parallel to the dilator and not perpendicular. Heat the glass in the center of the caudal end until it becomes white hot at which point take a small diameter glass rod, oriented parallel to the dilator and poke into this area, actually protruding into the dilator without touching any sides. As the lathe spins, the molten glass of the caudal end wraps around the rod and if the rod is quickly removed the flame cuts the rod from the dilator. As this happens, the wall thickness of the then-closed caudal end becomes thinner and thinner. Continue to poke the closed end in this manner until the glass is so thin that the hole 40 opens up and the flame is immediately removed from the art. Left is a hole or pressure port slightly smaller than the diameter of the tube.

For the inner lip 45, reposition the flame back to just slightly past perpendicular to the stent where the flame is angled inward toward the open end 40. Continue to use a needle point flame and we aim it directly at the last two millimeters of open caudal end 40 where the hole was just made. As the flame heats the caudal end, again the glass will condense inward and begin to close up the opening we created. When this opening is approximately ten millimeters smaller than the original diameter we remove the heat leaving a thick rim of glass 45 in FIG. 3 on the inner portion of the open caudal end 40.

With the retractor or caudal end completed, remove the disconnected raw glass left in the chuck at the caudal end and adjust the movable chuck to secure the caudal end in the chuck, making sure to protect the stent with a heat cloth placed over claws the clutch to prevent any scratches or nicks or reaction to the metal or the chuck. With the caudal end secured in this chuck, return the flame to the smallest point of the tapered distal end to create the hole 30 in FIG. 1. Repeat the same procedure used to create hole 40 in the caudal end. Use the needlelike flame to condense the glass and seal the tapered distal end. Widen the chucks to separate the stent from the remaining tube glass. Again, reposition the flame to be almost parallel to the distal end. Since this end is narrower than the caudal end use a much smaller needlelike flame to prevent overheating of the glass near the tip. As before, use a small glass rod to quickly poke into the distal end, repeating this motion until a hole is opened. Once the hole is created, we return the flame back to perpendicular. Using a very small flame, we focus the heat on the tip of the open hole to round off and polish the edge of the hole 30 without creating a thick inner lip like the caudal end.

When completed, the dilator is removed from the chuck, grabbed with protective gloves and placed in a kiln to anneal to relieve any internal stresses created during the process. Follow standard annealing guidelines for glass thicknesses.

For this invention, borosilicate glass is preferred for its non-porous surface and its overall durability and beneficial properties, but other nondeformable materials can be used such as wood, metals or plastic, and equivalents although less preferable, can be made from other materials such as silicone, and like materials. For these other materials, the same manufacturing process can be used but is not limited to these. Injection molding, mold forming, and basic carving are three widely used manufacturing examples that can produce equivalent shaped embodiments of the invention, but are not limited to just these processes.

By operation of the foregoing steps, an article of nondeformable material is made with integrally formed structural features that are useful as a vaginal dilator. The article of manufacture is comprised, in general, of three integral parts: a distal or insertion end, a dilator body, and a retractor or caudal end.

The insertion end of the dilator, in its preferred embodiment, has a generally frusto-conical shape, with arc-shaped lines forming the cone instead of straight lines. The arcs create a tapered, rather than a pure conical, shape to the body tapering down to the distal end. The frustum cuts off the apex of the conical end of the distal portion, and therein, is formed a port of predetermined size, or an opening therethrough, into the hollow body of the dilator.

The body of the preferred article is hollow, with openings at each end. Integrally formed in the walls of the body is at least one, circumferential notch. Each notch is formed in the body at a predetermined location between the distal end and the caudal end. The location is predetermined according to the circumferential size of the body. The circumferential notch, in one preferred embodiment, is a continuous depression around the body. However, the notch does not have to be continuous, and an equivalent structure would be a series of smooth-featured depressions in the body around its circumference and at the predetermined location.

The retraction or caudal end of the preferred embodiment of the dilator has several structural features: a pressure port into the hollow body that allows for air fluid communication with the opening in the proximal end, a hourglass neck that widens open to the caudal end forming a retractor, and within the retractor or caudal end portion an integrally formed inner rib or lip at or near the caudal end.

While the present invention has been described in terms of specific embodiments, it is to be understood that the invention is not limited to these disclosed embodiments. This invention may be embodied in many different forms and should not be construed as limited to the embodiments set forth herein; rather, these embodiments are provided by way of illustration only and so that this disclosure will be thorough, complete and will fully convey the full scope of the invention to those skilled in the art. Indeed, many modifications and other embodiments of the invention will come to mind of those skilled in the art to which this invention pertains, and which are intended to be and are covered by both this disclosure, the drawings and the claims.

I claim:

1. An article of manufacture useful as a manual dilator, comprising:
a unitary, tubular article having a distal end, a body and a caudal end, with said body having a predetermined circumferential girth tapering down to said distal end, and said distal end having an apex that defines a port into the tubular body, and said caudal end having a port into said tubular body and a retractor portion with an integrally formed lip.

2. The article of claim 1, further comprising at least one circumferential notch on said body.

3. The article of claim 2, wherein said notch is a predetermined location on said body.

4. The article of claim 3, wherein the location of said notch is predetermined by the circumferential girth of said body.

5. The article of claim 2 further comprising, between said body and said caudal end, an hourglass having a predetermined circumferential girth smaller than said body, and said caudal end having a predetermined circumferential girth larger than said hourglass, and the circumferential girth of said caudal end predetermined to be equal to or smaller than the girth of said body.

6. A manual dilator, comprising material integrally formed into a nondeformable dilator body having an open caudal end portion and a distal end portion, wherein said body is tubular having a first port formed in said open caudal end portion and a second port formed in said distal end portion, and an hourglass port between said body and said open caudal end portion, and a circumferential retractor lip formed inside said open caudal end portion.

7. The article of claim 6, said body having a predetermined circumferential girth tapering down to said second port in said distal end.

8. The article of claim 6, further comprising at least one circumferential notch on said body.

9. The article of claim 8, wherein said notch is a predetermined location on said body.

9. The article of claim 6, said caudal end portion further comprising a retractor that widens in circumferential girth from said hourglass port out to said open caudal end portion.
